(19) United States
(12) Patent Application Publication
(10) Pub. No.: US 2004/0249407 A1
(43) Pub. Date: Dec. 9, 2004

(54) Offset arteriotomy locator

(76) Inventor: Loran James Paprocki, St. Louis Park, MN (US)

Correspondence Address: David E. Bruhn, DORSEY & WHITNEY LLP, Intellectual Property Department, 50 South Sixth Street, Suite 1500, Minneapolis, MN, 55402-1498, US

(21) Appl. No.: 10/454,791

(22) Filed: Jun. 4, 2003

(51) Int. Cl. A61B 17/00

(52) U.S. Cl. 606/190

(57) ABSTRACT

An arteriotomy locator for use in a vascular procedure involving a sheath wherein the arteriotomy locator and sheath are inserted through the tissue of a medical patient and the sheath comprises a sheath wall and an angled tip including a leading edge and a trailing edge, and the arteriotomy locator comprises a distal section, a proximal section and a structural feature located between the sections, wherein the structural feature is adapted to provide a transition from the arteriotomy locator to the sheath. A method of using the arteriotomy locator is encompassed.

### BACKGROUND

The present invention relates to catheters and the like, and medical procedures involving catheters. More specifically, the present invention relates to vascular devices, including vascular wound closure devices, and structures and methods for minimizing insertion interference between an arteriotomy locator and the surrounding tissue of a puncture through which the vascular device passes.

Various catheterization procedures require the piercing of a vessel, for example, a blood vessel, arteries, or a body organ. Withdrawal of the catheter leaves a puncture wound that must be closed. For pierce or puncture wounds in blood vessels, traditional closure techniques involve withdrawal of the catheter and subsequent application of pressure to the area of the puncture to facilitate natural clotting. Such a technique is time consuming, and the time required and the results vary greatly from patient to patient.

The assignee of the present application owns patents and patent applications directed to vascular closure devices and their use. For example, U.S. Pat. Nos. 5,108,421, 5,192,300, 5,275,616, 5,478,352, 5,591,205, 6,601,602 and 5,716,375, all entitled “Insertion Assembly and Method of Inserting a Vessel Plug into the Body of a Patient,” disclose closure devices and methods of using them.

U.S. Pat. No. 5,306,254, which issued Apr. 26, 1994 and is incorporated herein by reference in its entirety, describes a vascular closure device and method involving introducing a mechanical seal through a sheath. Generally, the disclosed closure device enables sandwiching an arteriotomy between a bio-absorbable anchor and a collagen sponge, both of which dissolve after time. This technique consistently, reliably, and bio-mechanically seals the puncture allowing for faster recovery as well as providing for a faster completion of the procedure.

During some catheterization procedures, a sheath is inserted into an artery or other vessel and a given medical procedure, such as, for example, a cardiac catheterization, balloon angioplasty, angiographic dye injection or the like, is performed. Upon completion, a guidewire is inserted through the catheter sheath and into the artery or other body vessel. The sheath is then removed, leaving the guidewire in place.

The assignee's vascular closure device may then used to seal the artery puncture. More specifically, an arteriotomy locator is introduced into a sheath of the vascular closure device. The guidewire is then fed through the arteriotomy locator. The combined arteriotomy locator and sheath are then advanced into the artery through the existing puncture, guided by the guidewire.

To determine the position of the sheath during insertion, a detection orifice is provided in the arteriotomy locator, generally distal to the sheath tip. The orifice is in fluid communication with a proximal orifice (or other indicator) that is visible to the surgeon. The arteriotomy locator and sheath are advanced through the artery wall, until a “flashback” (e.g., blood flow) is detected in the proximal orifice indicating that the detection orifice is within the artery. It should be understood that the present invention need not be used exclusively in or on arteries; however, if it is being used on an artery, the blood flow may be pulsitile. When blood flow is detected, the sheath and arteriotomy locator are then retracted just until the flashback has ceased. This indicates that the detection orifice is aligned with the interior edge of the artery wall and the sheath has been retracted from the artery. As such, the arteriotomy locator and sheath are now in a known position relative to the artery. Both can be advanced a specific distance (e.g., 1-2 cm) and the surgeon is assured of proper placement of the arteriotomy locator and sheath relative to the artery. It is desirable to limit the advancement of the sheath so as to not pass the sheath too far into the artery. Some surgeons may repeat these steps (advancing and retracting) several time to obtain a comfort level with the position of the sheath.

As the sheath and arteriotomy locator advance into the puncture, generally thin, tough tissue, e.g., a vessel wall, at the edge of the puncture slides over or along the arteriotomy locator and sheath. The sheath tip has an edge that may catch the tissue, thereby impeding further insertion of the sheath and presenting the potential for tissue damage (e.g., damage to the vessel wall) or damage to the sheath tip. The potential for damage may be multiplied by the tendency of some surgeons to repeatedly advance and retract the sheath several times to ensure the proper placement of the sheath in the artery.

Interference between the edge of the sheath tip and the tissue is more likely where the edge is thick due to a thick tip wall. Manufacturing sheaths with the thinnest edges possible has been the industry's attempt at addressing the problem of interference between the edge of the sheath tip and tissue. However, because of material or manufacturing limitations, it is difficult to consistently produce sheaths with sufficiently thin edges. Durability is also adversely affected.

There is a need in the art for a device and method for minimizing interference between the edge of a sheath tip and the tissue surrounding a puncture.

### SUMMARY

In one embodiment, the present invention comprises an locator having a body comprising an offset to be positioned adjacent to a portion of a sheath for use with the locator.

In one embodiment, the present invention comprises an arteriotomy locator having a body comprising an offset to be positioned adjacent to at least a portion of a leading edge of a sheath being used with the arteriotomy locator.

In one embodiment, the present invention comprises an arteriotomy locator having a shaft which has been offset to cover at least a portion of a leading edge of a sheath being used with the arteriotomy locator. The outer diameter of the locator is not changed.

In one embodiment, the present invention comprises an arteriotomy locator having an offset portion for contacting at least a portion of a leading edge of a catheter being used with the arteriotomy locator, wherein the portions of the locator on either side of the offset portion have substantially the same outer diameter. In some embodiments, the offset portion covers all or a portion of the leading edge of a sheath being used with the locator. In embodiments in which a portion of the leading edge of a sheath is covered, then the trailing portion of the sheath tip generally passes a tissue easily regardless of the thickness of the sheath.

In one embodiment, the present invention comprises an arteriotomy locator having an offset for improving sheath tip transition by shrouding or covering a portion of a sheath tip while not increasing the outer diameter of the locator. In some embodiments, the offset is generally in proximity to the sheath tip, the body of the locator, in some instances, a generally tubular and cylindrical body, being shifted or stepped so that the inner and outer diameters are the same, but the axis of the locator is displaced, shifted or split into two parallel axes. In other words, at the offset, what would be the single, generally central longitudinal axis of the locator is laterally shifted or split to a generally parallel position, effectively giving the locator two generally parallel central longitudinal axes. The displacement or shift may be of any suitable dimension.

Advantageously, in one embodiment, the offset arteriotomy locator of the present invention avoids the need for a raised portion encircling or partially encircling the locator body. Such a raised portion or area would be a localized larger outer diameter that could create problems as a user tries to withdraw such a locator through a sheath. A locator with a raised portion might also require a correspondingly modified sheath, for example, to accommodate inserting or withdrawing the locator, to accommodate the locator when it is in the sheath, or to provide for sealing against or directing blood flow.

In one embodiment, the present invention comprises an arteriotomy locator for use in a vascular procedure involving a sheath comprising a sheath wall and an angled tip including a leading edge and a trailing edge, the arteriotomy locator comprising a distal section, a proximal section joining the distal section, and a structural feature located between the sections, wherein the structural feature is adapted to provide a transition between the arteriotomy locator and the sheath to protect tissue and/or the sheath as tissue slides along the Tdistal section of the arteriotomy locator and onto the sheath. A vascular procedure or method of using the arteriotomy locator is encompassed.

The present invention, in one embodiment, comprises an arteriotomy locator for use with a vascular procedure sheath wherein a sheath is inserted through the tissue of a medical patient and comprises a sheath wall and an angled tip including a leading edge and a trailing edge. The arteriotomy locator comprises a distal section, a proximal section joining the distal section, and a structural feature located between said sections. The structural feature is adapted to be positioned adjacent to, and/or at least partially contact, cover or shroud the leading edge to reduce the edge/tissue interference or interaction as tissue slides along the distal section of the arteriotomy locator and up onto the sheath, thereby protecting the tissue and the sheath tip. The structural feature also provides for a relatively smoother transition in the opposite direction, i.e., as tissue slides from the sheath onto the locator. In one embodiment of the arteriotomy locator, the structural feature is an offset comprising a forward sloping surface and a rearward sloping surface, the forward sloping surface adapted to be positioned adjacent to, to shroud or cover or generally cover, the leading edge. In another embodiment, the structural feature is an offset, the proximal section includes a longitudinal axis, and the distal section includes a longitudinal axis generally parallel to the longitudinal axis of the proximal section. In another embodiment, the structural feature is a groove, which encompasses at least a portion of the outer circumference of the arteriotomy locator, and which includes a sloped or curved portion to matingly complement or correspond to the angled tip of the sheath, and is thus adapted to shroud or cover at least a portion of the leading edge.

In another embodiment, the present invention is a vascular procedure device comprising a sheath and an arteriotomy locator. The sheath has an angled tip including a leading edge and a trailing edge. The arteriotomy locator comprises a distal end, a proximal end and an offset disposed between said ends. The offset comprises a forward sloping surface and a rearward sloping surface. The forward sloping surface is adapted to transition, generally gradually and smoothly, between the arteriotomy locator and the sheath to minimize interference between the leading edge and tissue as the sheath and locator are advanced through a tissue.

In another embodiment, the present invention is an arteriotomy locator for use with a vascular procedure sheath wherein the sheath comprises a tip, which includes a leading edge and a trailing edge. The arteriotomy locator comprises a proximal section joined to a distal section. The proximal section has a first longitudinal axis and the distal section has a second longitudinal axis offset parallel from the first longitudinal axis.

While multiple embodiments of the arteriotomy locator and methods of the present invention are disclosed, still other embodiments will become apparent to those skilled in the art from the following detailed description, which shows and describes illustrative embodiments of the invention. As will be realized, the invention is capable of modifications in various aspects, all without departing from the spirit and scope of the present invention. Accordingly, the drawings and detailed description are illustrative, not restrictive.

### BRIEF DESCRIPTION OF THE DRAWINGS

FIG. 1 is a side schematic view of a sheath for the catheter device of the subject invention.

FIG. 2 is a side schematic view of an arteriotomy locator for the catheter device of the subject invention.

FIG. 3 is a side schematic view of the catheter device of the subject invention with the arteriotomy locator of FIG. 2 located within the sheath of FIG. 1.

FIG. 4 an enlarged side cross-section schematic view of the distal end of the catheter device illustrated in FIG. 3, according to one embodiment of the invention, and showing the leading and trailing edges of the sheath tip and the offset in the arteriotomy locator.

FIG. 5 an enlarged side cross-section schematic view of the distal end of the catheter device illustrated in FIG. 3, according to another embodiment of the invention, and showing the leading and trailing edges of the sheath tip and the groove in the arteriotomy locator.

FIG. 6 an enlarged side cross-section schematic view of the distal end of the catheter device illustrated in FIG. 3, according to another embodiment of the invention, and showing the leading and trailing edges of the sheath tip and the groove in the arteriotomy locator.

### DETAILED DESCRIPTION

The present invention comprises an arteriotomy locator for use with catheters, catheter devices and/or catheterization systems and procedures, including with a sheath of a vascular wound closure device. The arteriotomy locator is also useable with catheter sheaths used in other various vascular and minimally invasive medical and surgical procedures, such as catheterizations and laparoscopy.

In one embodiment, the arteriotomy locator has a structure, which may comprise an offset, variance, discontinuity, or transition in or on its exterior surface wherein the longitudinal axis of the arteriotomy locator shifts to another parallel plane longitudinally along the arteriotomy locator and across the region having the variance, discontinuity, or transition characterization. In other embodiments, the arteriotomy locator has a variance, discontinuity, or transition in its exterior surface wherein the outer diameter of the arteriotomy locator changes (e.g., decreases) longitudinally along the arteriotomy locator and continuously or discontinuously across the variance, discontinuity, or transition. In embodiments of the arteriotomy locator, the variance, discontinuity, or transition in the exterior surface of the arteriotomy locator forms a structural feature that is integral with, attached to or formed in the exterior surface and is adjacent to, contacts, shrouds or covers at least a portion of the leading edge of a sheath tip to minimize insertion interference between the sheath tip and the tissue (e.g., blood vessel wall) surrounding a puncture through which the sheath passes.

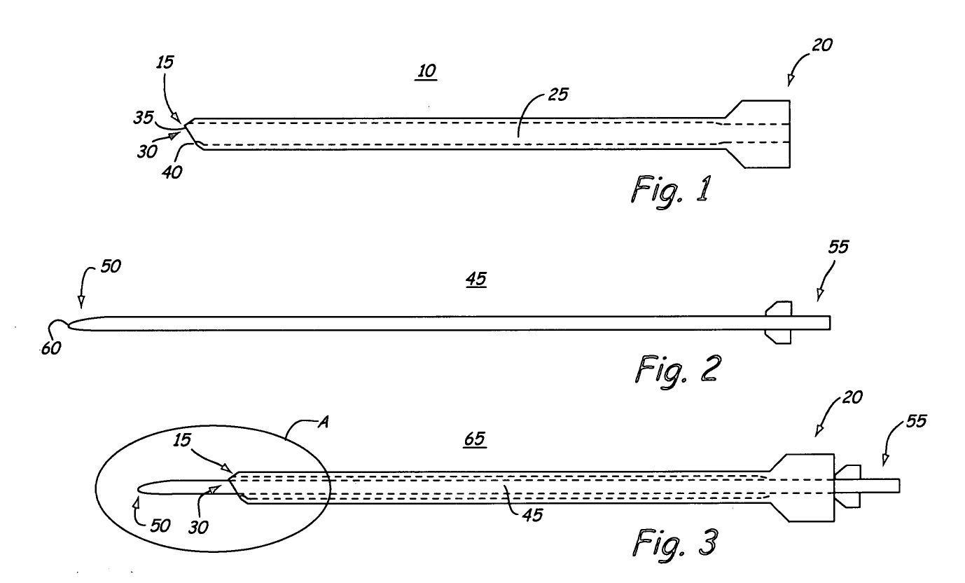

FIG. 1 is a side schematic view of a sheath 10 for the catheter device of the present invention. As shown in FIG. 1, the sheath 10 has a distal end 15 (i.e., the end that would face a patient) and a proximal end 20 (i.e., the end that would face the physician). A channel or bore 25 for receiving an arteriotomy locator runs through the sheath 10 from the proximal end 20 to the distal end 15. The distal end 15 of the sheath 10 has an angled or slanted tip 30. In other words, the angled sheath tip 30 has a leading edge 35 and a trailing edge 40.

FIG. 2 is a side schematic view of an arteriotomy locator 45 for use with, for example, the catheter device of the subject invention. As indicated in FIG. 2, the arteriotomy locator 45 also has a distal end 50 and a proximal end 55. The distal end 50 of the arteriotomy locator 45 has a rounded tapered tip 60. In one embodiment, the arteriotomy locator 45 has a hollow channel running its entire length. Thus, the arteriotomy locator 45 has an outer diameter and an inner diameter.

FIG. 3 is a side schematic view of the catheter device 65 of the subject invention with the arteriotomy locator 45 located within the channel 25 of the sheath 10. Area A of FIG. 3 encompasses the distal end of the catheter device 65 (i.e., the distal end of the 15 sheath and the distal end 50 of the arteriotomy locator 45).

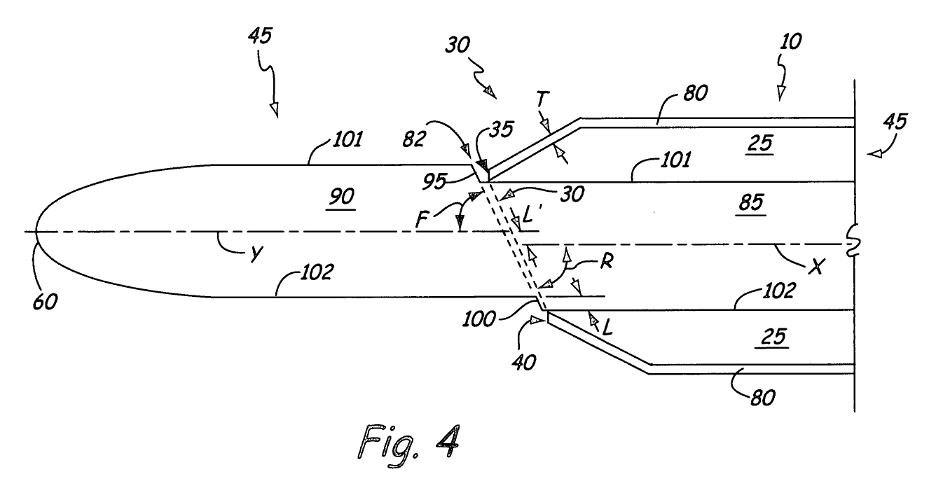

FIG. 4 is an enlarged side cross-sectional schematic view of area A of FIG. 3 for one embodiment of the invention. As illustrated in FIG. 4, the sheath wall 80 is generally parallel to the outer surface of arteriotomy locator 45 until the wall nears the angled sheath tip 30. At that point, the sheath wall 80 angles towards the outer surface of the arteriotomy locator 45, forming the angled sheath tip 30, which has a leading edge 35 and a trailing edge 40. As shown in FIG. 4, the leading edge 35 of the angled sheath tip 30 abuts against the exterior surface of the arteriotomy locator 45. Likewise, the trailing edge 40 of the angled sheath tip 30 abuts against the exterior surface of the arteriotomy locator 45.

As indicated in FIG. 4, the arteriotomy locator 45 has an offset 82 along its length. The offset is adapted to be positioned adjacent to or near the angled sheath tip 30 when the locator is in use. Thus, the arteriotomy locator 45 can be said to have at least two general sections, the proximal section 85, which is on the proximal side of the offset 82, and the distal section 90, which is on the distal side of the offset 82. As shown in FIG. 4, the arteriotomy locator 45 is stepped at the offset 82 so the longitudinal axis X of the proximal section 85 is offset from and generally parallel to the longitudinal axis Y of the distal section 90 while the outer and inner diameters of the proximal section 85 are equal to the outer and inner diameters of the distal section 90.

As illustrated in FIG. 4, the offset 82 is angled/slanted to matingly correspond with the angled/slanted sheath tip 30. Thus, the offset 82 has a forward sloping surface 95 and a rearward sloping surface 100. In other words, one would travel up the forward sloping surface 95 when traveling along the arteriotomy locator's top surface 101 from the proximal section 85 to the distal section 90. Similarly, one would travel down the rearward sloping surface 100 when traveling along the arteriotomy locator's bottom surface 102 from the distal section 90 to the proximal section 85. This configuration is advantageous for at least two reasons. The sloping surfaces 95, 100 allow the leading and trailing edges 35, 40 to smoothly displace along the outer surface of the arteriotomy locator 45 from the proximal section 85 to the distal section 90, and vice versa, as the arteriotomy locator 45 is extended from or retracted into the sheath 10.

Also, the forward sloping surface 95 serves to shroud, or cover, the leading edge 35 of the angled tip 30 to help prevent it from interfering with tissue sliding along the distal section 90 of the arteriotomy locator 45 and onto the sheath 10. The protective shrouding effect of the offset 82 minimizes the insertion interference between the angled tip 30 and tissue, even when the sheath wall 80 is thick. Further, though the trailing edge 40 is not shrouded or covered, experience has shown that the trailing edge 40 passes easily through a penetration in tissue without significant interference when the leading edge 35 has preceded the trailing edge 40 through the penetration. Thus, the offset 82 improves sheath tip transition by shrouding a portion of the sheath tip 30 without increasing the outer diameter of the arteriotomy locator 45. This is the case even when the sheath tip 30 has a thick sheath wall 80.

In one embodiment, the forward sloping surface 95 forms an angle F with the longitudinal axis Y of approximately five to approximately 85 degrees and the rearward sloping surface 100 forms an angle R with the longitudinal axis X of approximately five to approximately 85 degrees. In another embodiment, the forward sloping surface 95 forms an angle F with the longitudinal axis Y of approximately 15 to approximately 75 degrees and the rearward sloping surface 100 forms an angle R with the longitudinal axis X of approximately 15 to approximately 75 degrees. In yet another embodiment, the forward sloping surface 95 forms an angle F with the longitudinal axis Y of approximately 30 to approximately 60 degrees and the rearward sloping surface 100 forms an angle R with the longitudinal axis X of approximately 30 to approximately 60 degrees.

In one embodiment, the length L of each sloping surface 95, 100 (wherein the length L is measured parallel to a sloping surface) is approximately 0.25 to approximately 4.0 times the thickness T of the sheath wall 80. In another embodiment, the length L of each sloping surface 95, 100 is approximately 0.50 to approximately 3.0 times the thickness T of the sheath wall 80. In yet another embodiment, the length L of each sloping surface 95, 100 is approximately 0.75 to approximately 2.0 times the thickness T of the sheath wall 80.

In one embodiment, the offset length L′ between the longitudinal axes X, Y (wherein the length is measured parallel to the slope of the offset 82) is approximately 0.25 to approximately 4.0 times the thickness T of the sheath wall 80. In another embodiment, the offset length L′ is approximately 0.50 to approximately 3.0 times the thickness T of the sheath wall 80. In yet another embodiment, the offset L′ is approximately 0.75 to approximately 2.0 times the thickness T of the sheath wall 80. All lengths, angles and dimensions may be varied within suitable ranges.

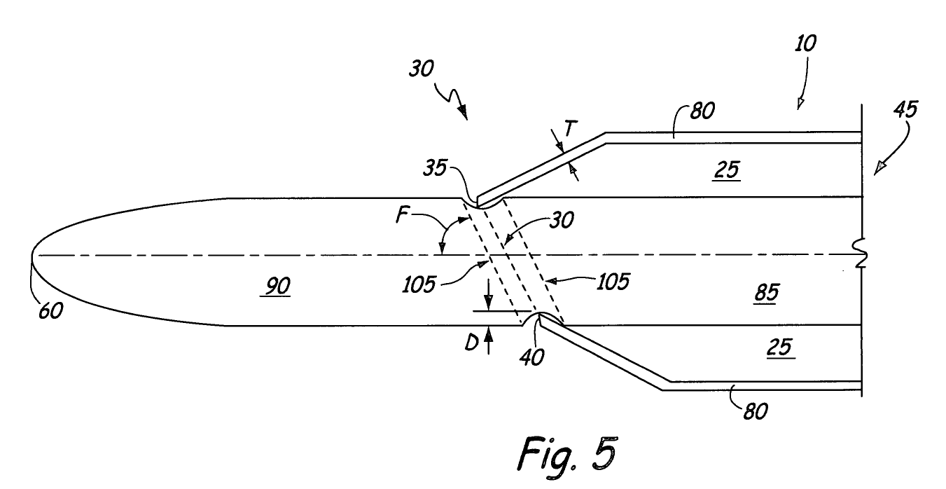

FIG. 5 is an enlarged side cross-sectional schematic view of area A of FIG. 3 for another embodiment of the invention. As shown in FIG. 5, the sheath 10 has the same configuration as previously described with reference to FIG. 4, but the arteriotomy locator 45 has a groove 105 about its outer circumference, near the proximity of the angled sheath tip 30, for shrouding both the leading and trailing edges 35, 40. Again, the arteriotomy locator 45 can be said to have two sections, the proximal section 85, which is on the proximal side of the groove 105, and the distal section 90, which is on the distal side of the groove 105.

As shown in FIG. 5, the groove 105 encircles the entire outer circumference of the arteriotomy locator 45 and is angled/slanted forward to matingly correspond with the angled sheath tip 30, thereby providing a recess that shrouds at least a portion of both the leading and trailing edges 35, 40 from interfering with tissue sliding along the distal section 90 of the arteriotomy locator 45 and up onto the sheath 10. The shrouding effect of the groove 105 minimizes the insertion interference between the angled tip 30 and tissue, even when the sheath wall 80 is thick.

As illustrated in FIG. 5, the groove 105 has a curved profile. The curved profile allows the leading and trailing edges 35, 40 to smoothly displace along the outer surface of the arteriotomy locator 45 from the proximal section 85 to the distal section 90, and vice versa, as the arteriotomy locator 45 is extended from or retracted into the sheath 10.

In one embodiment, the groove 105 is sloped/slanted forward so as to form an angle F with the longitudinal axis of the arteriotomy locator 45 of approximately five to approximately 85 degrees. In another embodiment, the groove 105 is sloped forward so as to form an angle F with the longitudinal axis of the arteriotomy locator 45 of approximately 15 to approximately 75 degrees. In yet another embodiment, the groove 105 is sloped forward so as to form an angle F with the longitudinal axis of the arteriotomy locator 45 of approximately 30 to approximately 60 degrees.

In one embodiment, the depth D of the groove 105 is approximately 0.25 to approximately 4.0 times the thickness T of the sheath wall 80. In another embodiment, the depth D of the groove 105 is approximately 0.50 to approximately 3.0 times the thickness T of the sheath wall 80. In yet another embodiment, the depth D of the groove 105 is approximately 0.75 to approximately 2.0 times the thickness T of the sheath wall 80. Again, all stated depths, lengths, angles and dimensions may be varied as suitable.

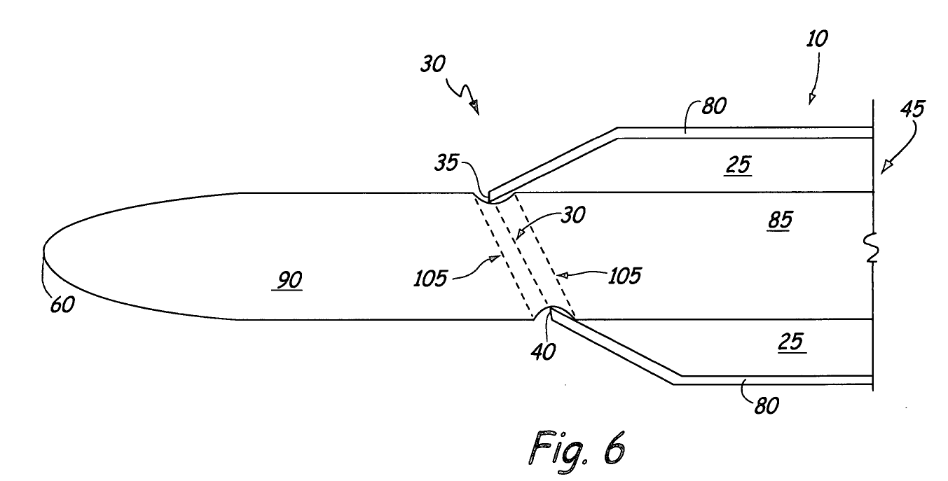

FIG. 6 is an enlarged side cross-sectional schematic view of area A of FIG. 3 for another embodiment of the invention. The embodiment shown in FIG. 6, is similar to the embodiment described in FIG. 5, except the groove 105 only encircles a portion of the outer circumference of the arteriotomy locator 45. As shown in FIG. 6, the groove 105 shrouds the leading edge 35 while leaving the trailing edge 40 exposed. However, as discussed above, though the trailing edge 40 is not shrouded, experience has shown that the trailing edge 40 passes easily through a penetration in tissue without significant interference when the leading edge 35 has preceded the trailing edge 40 through the penetration. Thus, the groove 105, whether it encompasses the entire outer circumference of the arteriotomy locator 45, as shown in FIG. 5, or encompasses less than the entire outer circumference of the arteriotomy locator 45, as shown in FIG. 6, improves sheath tip transition by shrouding at least a portion of the sheath tip 30 without increasing the outer diameter of the arteriotomy locator 45. This is the case even when the sheath tip 30 has a thick sheath wall 80.

The present invention is useful for any type of catheter and medical procedure involving a catheter or the like, including catheters and procedures involving body organs or blood vessels, including arteries. In one context, the present invention is used with an blood vessel puncture sealing apparatus, however, such use is merely meant to be exemplary and not limiting. Although the present invention has been described with reference to preferred embodiments, persons skilled in the art will recognize that changes may be made in form and detail without departing from the spirit and scope of the invention.

We claim:

1. An arteriotomy locator for use with a vascular procedure sheath wherein the arteriotomy locator and sheath are inserted into the lumen of a vessel of a medical patient and the sheath comprises a sheath wall and an angled tip including a leading edge and a trailing edge, the arteriotomy locator comprising:
a distal section;
a proximal section generally adjoining the distal section; and
a structural feature located generally between said sections, the structural feature adapted to transition from the arteriotomy locator to the sheath.

2. The arteriotomy locator of claim 1 wherein the structural feature is an offset comprising a surface adapted to shroud at least a portion of the leading edge.

3. The arteriotomy locator of claim 2 wherein the surface has a length of approximately 0.25 to approximately 4.0 times the thickness of the sheath wall.

4. The arteriotomy locator of claim 1 wherein the structural feature is an offset, the proximal section having a first longitudinal axis, and the distal section having a second longitudinal axis generally parallel to the first longitudinal axis.

5. The arteriotomy locator of claim 4 wherein the offset length between the longitudinal axes is approximately 0.25 to approximately 4.0 times the thickness of the sheath wall.

6. The arteriotomy locator of claim 4, the offset further comprising a sloping surface adapted to be positioned adjacent to the leading edge, wherein the sloping surface forms an angle with the second longitudinal axis of approximately five to approximately 85 degrees.

7. The arteriotomy locator of claim 1 wherein the structural feature is a groove, which encompasses at least a portion of the outer circumference of the arteriotomy locator, at least a portion of the groove adapted to complement a portion of the angled tip of the sheath to provide a transition between the locator and the leading edge.

8. The arteriotomy locator of claim 7 wherein the groove encircles the outer circumference of the arteriotomy locator and provides a transition between the locator and the leading edge and trailing edge.

9. The arteriotomy locator of claim 7 wherein the groove has a depth of approximately 0.25 to approximately 4.0 times the thickness of the sheath wall.

10. The arteriotomy locator of claim 7 wherein the groove is sloped forward so as to form an angle with the longitudinal axis of the arteriotomy locator of approximately five to approximately 85 degrees.

11. An arteriotomy locator comprising a shaft which has been offset to cover at least a portion of a leading edge of a sheath being used with the arteriotomy locator, wherein the shaft has a single outer diameter.
